(19) United States
(12) Patent Application Publication
(10) Pub. No.: US 2026/0020902 A1
(43) Pub. Date: Jan. 22, 2026

(54) LEAFLET MODIFICATION DEVICE WITH INDEPENDENTLY ACTUATED PIERCING ELECTRODE AND LACERATING ELECTRODE

(71) Applicant: BOSTON SCIENTIFIC SCIMED, INC., MAPLE GROVE, MN (US)

(72) Inventors: Eric Michael Petersen, Maple Grove, MN (US); Joel T. Eggert, Plymouth, MN (US); Daniel Presteng, Corcoran, MN (US); Joseph Edward Adriaens, Elk River, MN (US); Philip Andrew Litecky, Forest Lake, MN (US); Lauren Koon, Etobicoke (CA)

(73) Assignee: BOSTON SCIENTIFIC SCIMED, INC., MAPLE GROVE, MN (US)

(21) Appl. No.: 19/270,851

(22) Filed: Jul. 16, 2025

Related U.S. Application Data
(60) Provisional application No. 63/674,000, filed on Jul. 22, 2024.

(51) Int. Cl. A61B 18/14; A61B 18/00

(52) U.S. Cl. CPC A61B 18/1492; A61B 2018/00184; A61B 2018/00369; A61B 2018/00601

(57) ABSTRACT

A medical device for cutting valve leaflets includes an elongate shaft that extends proximally from a deflectable distal region, where the deflectable distal region includes a distal end and the deflectable distal region includes a biased configuration. A piercing is electrode disposed relative to the distal end and is adapted to pierce through a valve leaflet. A lacerating electrode is spaced from the piercing electrode and is adapted to lacerate the valve leaflet subsequent to the piercing electrode piercing the valve leaflet. The piercing electrode and the lacerating electrode are independently energized.

### CROSS REFERENCE TO RELATED APPLICATIONS

This application claims the benefit of priority of U.S. Provisional Application No. 63/674,000 filed Jul. 22, 2024, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, for example, intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical device for cutting valve leaflets. The medical device includes an elongate shaft extending proximally from a deflectable distal region having a distal end. The deflectable distal region has a biased configuration. A piercing electrode is disposed relative to the distal end and is adapted to pierce through a valve leaflet. A lacerating electrode is spaced and electrically insulated from the piercing electrode and is adapted to lacerate the valve leaflet subsequent to the piercing electrode piercing the valve leaflet. The piercing electrode and the lacerating electrode are independently energized.

Alternatively or additionally, the medical device may further include a handle that is secured to a proximal region of the elongate shaft.

Alternatively or additionally, the piercing electrode may be electrically coupled with the handle such that the piercing electrode can be selectively coupled with an RF energy source.

Alternatively or additionally, the lacerating electrode may extend proximally to the handle and may be electrically coupled with the handle such that the lacerating electrode can be selectively coupled with an RF energy source.

Alternatively or additionally, the handle may further include a deflection actuation member.

Alternatively or additionally, the lacerating electrode may be coupled with the deflection actuation member such that actuating the deflection actuation member provides a tensile force on the lacerating electrode that causes the distal region to deflect from its biased configuration.

Alternatively or additionally, the biased configuration may correspond to the lacerating electrode being at least substantially parallel with the deflectable distal region

Alternatively or additionally, the medical device may further include an elongate slot that is formed within an outer surface of the deflectable distal region.

Alternatively or additionally, a portion of the lacerating electrode passing the elongate slot may remain within the lumen when the distal region is in the biased configuration.

Alternatively or additionally, the portion of the lacerating electrode passing the elongate slot may extend outside of the lumen when the distal region is deflected from the biased configuration.

Another example may be found in a medical device. The medical device includes an elongate shaft that extends proximally from a distal region. The distal region includes a distal end and an elongate slot that extends axially along an outer surface of the distal region and terminates proximally of the distal end. The distal region is deflectable from a biased configuration. A first electrode is disposed relative to the distal end. A second electrode is spaced from the first electrode and remains within the elongate slot when the distal region is in the biased configuration. The second electrode extends out of the elongate slot when the distal region is deflected from the biased configuration. Only one of the first electrode and the second electrode are energized at a time.

Alternatively or additionally, providing a tensile force on the second electrode may cause the distal region to deflect from the biased configuration.

Alternatively or additionally, the biased configuration may correspond to the second electrode being at least substantially parallel with a longitudinal axis of the elongate shaft.

Alternatively or additionally, the deflectable region may be adapted to be deflected to a configuration in which the second electrode extends at an angle between 45 degrees and 135 degrees with respect to the longitudinal axis of the elongate shaft.

Alternatively or additionally, the second electrode is not energized when the first electrode is energized.

Alternatively or additionally, the first electrode is not energized when the second electrode is energized.

Alternatively or additionally, the first electrode may be adapted to form a hole within a valve leaflet that the distal region can be advanced through.

Alternatively or additionally, the second electrode may be adapted to be pulled proximally through the valve leaflet subsequent to advancing the distal region through the hole in order to lacerate the valve leaflet.

Another example may be found in a medical device. The medical device includes an elongate shaft that extends proximally from a distal region to a proximal region. The distal region includes a distal end and an elongate slot that extends axially along an outer surface of the distal region and terminates proximally of the distal end. The distal region is deflectable from a biased configuration. A first electrode is disposed relative to the distal end. A second electrode is spaced from the first electrode and remains within the elongate slot when the distal region is in the biased configuration. The second electrode extends out of the elongate slot when the distal region is deflected from the biased configuration. A handle is secured to the proximal region and is adapted to selectively connect only one of the first electrode and the second electrode to a source of RF energy at a time.

Alternatively or additionally, the handle may further include a deflection actuation mechanism that may be adapted to apply a tensile force to the second electrode.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing a replacement heart valve implant positioned within a native valve annulus of a heart;

FIG. 2 is a schematic view of an illustrative medical device;

FIG. 3 is a schematic view of a portion of the illustrative medical device of FIG. 2;

FIG. 4A is a schematic view of a portion of the illustrative medical device of FIG. 2, showing a first electrode being energized while a second electrode is not energized;

FIG. 4B is a schematic view of a portion of the illustrative medical device of FIG. 2, showing a second electrode being energized while a first electrode is not energized;

FIG. 5 is a schematic view of a distal region of the illustrative medical device of FIG. 2, shown with the distal region in a biased configuration; and

FIG. 6 is a schematic view of the distal region of the illustrative medical device of FIG. 2, shown with the distal region in a configuration in which the distal region has been deflected from the biased configuration.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine tissue, or may be polymeric. In some instances, artificial valve leaflets may be made of polymers such as Dacron or Gore-Tex. As discussed here, reference to a valve leaflet may refer to either a native valve leaflet or an artificial valve leaflet.

As noted, the medical devices described herein may be used in lacerating valve leaflets regardless of whether the valve leaflets are native valve leaflets or artificial valve leaflets. In some cases, the native valve leaflets may be lacerated prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to lacerate one or more of the artificial valve leaflets within the previously implanted artificial heart valve before implanting the replacement artificial heart valve within the previously implanted artificial heart valve. In some cases, lacerating one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

In some instances, a medical device may be used for cutting valve leaflets, including native valve leaflets as well as artificial valve leaflets of a previously implanted artificial heart valve. The medical device includes an elongate shaft that extends proximally from a deflectable distal region. The deflectable distal region includes a distal end and has a biased configuration. A piercing electrode is disposed relative to the distal end and is adapted to pierce through a valve leaflet. A lacerating electrode is spaced from the piercing electrode and is adapted to lacerate the valve leaflet subsequent to the piercing electrode piercing the valve leaflet. The piercing electrode and the lacerating electrode are independently energized.

In some cases, the medical device may further include a handle that is secured to a proximal region of the elongate shaft. The piercing electrode may be electrically coupled with the handle such that the piercing electrode can be selectively coupled with an RF (radiofrequency) energy source. In some cases, the lacerating electrode may extend proximally to the handle and may be electrically coupled with the handle such that the lacerating electrode can be selectively coupled with an RF energy source. The handle may further include a deflection actuation member. In some cases, the lacerating electrode may be coupled with the deflection actuation member such that actuating the deflection actuation member may provide a tensile force on the lacerating electrode that causes the distal region to deflect from its biased configuration.

In some cases, the biased configuration may correspond to the lacerating electrode being at least substantially parallel with the deflectable distal region. The medical device may further include an elongate slot that is formed within an outer surface of the deflectable distal region. In some cases, a portion of the lacerating electrode passing the elongate slot may remain within the lumen when the distal region is in the biased configuration. The portion of the lacerating electrode passing the elongate slot may extend outside of the lumen when the distal region is deflected from the biased configuration.

In some instances, a medical device may include an elongate shaft that extends proximally from a distal region. The distal region includes a distal end and an elongate slot that extends axially along an outer surface of the distal region. The elongate slot terminates proximally of the distal end. The distal region is deflectable from a biased configuration. A first electrode is disposed relative to the distal end. A second electrode is spaced from the first electrode. The second electrode remains within the elongate slot when the distal region is in the biased configuration and extends out of the elongate slot when the distal region is deflected from the biased configuration. Only one of the first electrode and the second electrode are energized at a time.

In some cases, providing a tensile force on the second electrode may cause the distal region to deflect from the biased configuration. The biased configuration may correspond to the second electrode being at least substantially parallel with a longitudinal axis of the elongate shaft. In some cases, the deflectable region may be adapted to be deflected to a configuration in which the second electrode extends at an angle between about 45 degrees to about 135 degrees with respect to the longitudinal axis of the elongate shaft. The second electrode is not energized when the first electrode is energized. The first electrode is not energized when the second electrode is energized. In some cases, the first electrode may be adapted to form a hole within a valve leaflet that the distal region can be advanced through. In some cases, the second electrode may be adapted to be pulled proximally through the valve leaflet subsequent to advancing the distal region through the hole in order to lacerate the valve leaflet.

In some instances, a medical device may include an elongate shaft that extends proximally from a distal region. The distal region includes a distal end and an elongate slot that extends axially along an outer surface of the distal region. The elongate slot terminates proximally of the distal end. The distal region is deflectable from a biased configuration. A first electrode is disposed relative to the distal end. A second electrode is spaced from the first electrode. The second electrode remains within the elongate slot when the distal region is in the biased configuration and extends out of the elongate slot when the distal region is deflected from the biased configuration. A handle is secured to the proximal region and is adapted to selectively connect only one of the first electrode and the second electrode to a source of RF energy at a time. In some cases, the handle may further include a deflection actuation mechanism that is adapted to apply a tensile force to the second electrode.

As noted, the medical devices described herein may be used in lacerating valve leaflets regardless of whether the valve leaflets are native valve leaflets or artificial valve leaflets. In some cases, the native valve leaflets may be lacerated prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to lacerate one or more of the artificial valve leaflets within the previously implanted artificial heart valve before implanting the replacement artificial heart valve within the previously implanted artificial heart valve. In some cases, lacerating one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

In some instances, the medical devices described herein may be used in other medical procedures. As an example, the medical devices described herein may replace a guidewire in performing a retrograde aortic LAMPOON (Laceration of the Anterior Mitral leaflet to Prevent Outflow Obstruction) procedure. In this procedure, the medical device is advanced over the anterior leaflet and a piercing electrode is electrified. The leaflet is lacerated in a “top down” approach. The medical devices described herein may replace a guidewire in performing a trans-septal LAMPOON procedure in which the medical device is advanced over the anterior leaflet and a piercing electrode is electrified. The leaflet is lacerated in a “bottom up” approach. The medical devices described herein may replace a guidewire in performing a SESAME (SEptal Scoring Along the Midline Endocardium) procedure. A piercing electrode may be used to burrow through the endocardium. A lacerating electrode may be used to score the septum.

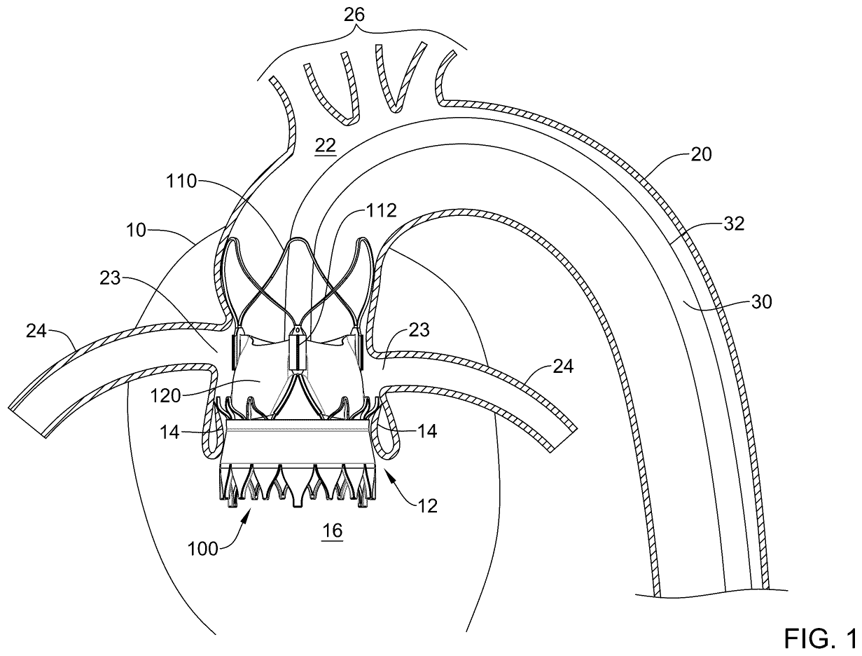

FIG. 1 is a schematic partial cut-away view of a portion of a patient's heart 10 including an aortic valve 12 having native valve leaflets 14 disposed within and/or extending from the native valve annulus, a left ventricle 16, and certain connected vasculature, such as an aorta 20 connected to the aortic valve 12 of the patient's heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient. In some cases, the following discussion may apply to both catheter-delivered cardiac valves and surgical cardiac valves, as well as to removing problematic tissue from native anatomy.

FIG. 1 further illustrates selected aspects of a replacement heart valve implant 100 positioned within the aortic valve 12 and/or the native valve annulus of the aortic valve 12. Some non-limiting examples of the replacement heart valve 100 may include the ACURATE NEO2™ the ACURATE PRIME™, and/or family members thereof from Boston Scientific. It should be appreciated that the replacement heart valve implant 100 can be any type of replacement heart valve (e.g., a mitral valve, an aortic valve, etc.). In use, the replacement heart valve implant 100 may be implanted (e.g., such as through transcatheter delivery) in the aortic valve 12 of the heart 10. The replacement heart valve implant 100 can be configured to allow one-way flow through the replacement heart valve implant 100 from an inflow end to an outflow end.

The replacement heart valve implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some instances, the expandable framework 110 may be self-expanding. In some instances, the expandable framework 110 may be self-biased toward the radially expanded configuration. In some cases, the expandable framework 110 may be mechanically expandable. As an example, the expandable framework 110 may be balloon expandable.

In some instances, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures 112. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some cases, the plurality of valve leaflets 120 may include a polymer such as a thermoplastic polymer. In some cases, the plurality of valve leaflets 120 may include at least 50 percent by weight of a polymer. In some instances, the plurality of valve leaflets 120 may be formed from porcine pericardium, bovine pericardium, or other tissue. Other configurations and/or materials are also contemplated.

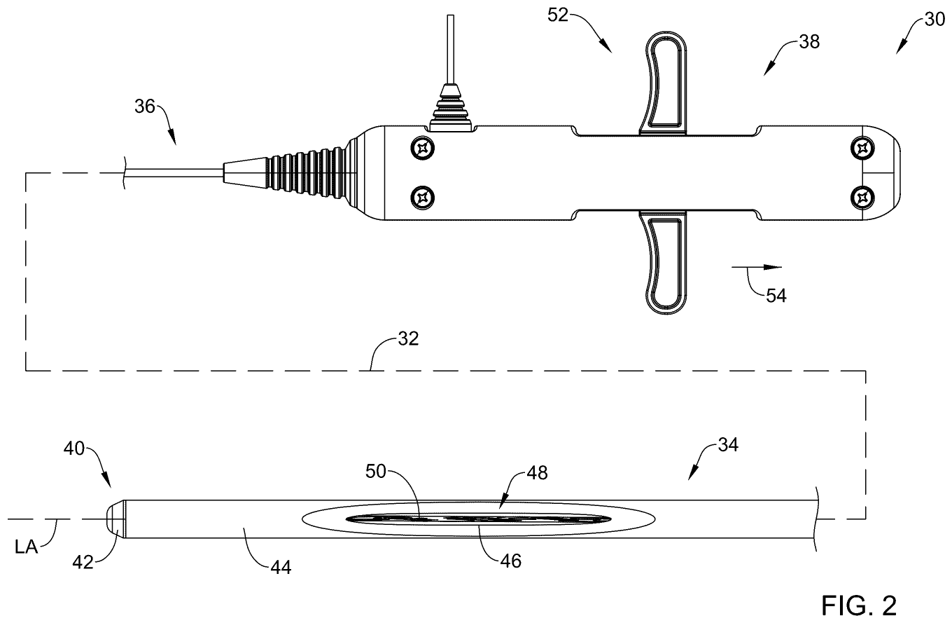

An example medical device 30 is shown in FIG. 1, extending through the aorta 20 and through the replacement heart valve 100. FIG. 2 is a schematic view of the medical device 30. It will be appreciated that the scale of FIG. 2 may not be constant, and may vary along the length of FIG. 2. The medical device 30 includes an elongate shaft 32 that extends from a distal region 34 to a proximal region 36. In some cases, a handle 38 may be secured relative to the proximal region 36. As will be discussed, the distal region 34 may be considered as being a deflectable distal region 34. In some cases, the distal region 34 includes a distal end 40. In some cases, the medical device 30 includes a first electrode 42 that is disposed at the distal end 40. As an example, the first electrode 42 may be considered as being a piercing electrode. In some cases, the first electrode 42 is stationary relative to the distal end 40. In some cases, the first electrode 42 may be axially translatable relative to the distal end 40. As an example, the first electrode 42 may be axially advanceable relative to the distal end 40, and can be extended for use and withdrawn into the distal end 40 when not in use.

In some cases, the distal region 34 of the elongate shaft 32 has an outer surface 44 that extends along the elongate shaft 32. As shown, an elongate slot 46 may be formed within the outer surface 44. The elongate slot 46 may extend in a direction parallel with a longitudinal axis LA and exposes a lumen 48 extending within the distal region 34 of the elongate shaft 32. In some cases, as shown, a second electrode 50 may be seen as extending within the lumen 48. The second electrode 50 may be axially spaced from the first electrode 42 and may be electrically insulated from the first electrode 42. The first electrode 42 and the second electrode 50 may form or be part of two electrically independent pathways. As shown, the distal region 34 of the elongate shaft 32 may be considered as being in a biased configuration, meaning that absent external forces, this is the configuration that the distal region 34 of the elongate shaft 32 will achieve. In some cases, as will be discussed, the distal region 34 of the elongate shaft 32 may be deflected from the biased configuration as part of actuating and then energizing the second electrode 50.

In some cases, the handle 38 may include electrical connections (not shown) for connecting a source of RF energy to the handle 38. In some cases, the handle 38 may itself incorporate a source of RF energy. The source of RF energy may be used to selectively energize either the first electrode 42 or the second electrode 50. As an example, the source of RF energy may include a commercially available RF generator such as those indicated for cutting soft tissue. Suitable RF generators include but are not limited to VALLEYLAB™ electrosurgical units and those available from Baylis Medical™ such as the Baylis Medical RFP-100A. In some cases, a non-RF energy source may be used. When the first electrode 42 is energized, the second electrode 50 is not energized. When the second electrode 50 is energized, the first electrode 42 is not energized. This allows a greater energy density for the electrode that is energized, and reduces the relative energy that would otherwise be lost to blood, rather than the target tissue or material such as native or artificial valve leaflets. In some cases, selective energization of either the first electrode 42 or the second electrode 50 can help prevent inadvertent cutting or other damage to non-target structures.

The handle 38 includes a deflection actuation member 52 that may be grasped by a user holding the handle 38. The deflection actuation member 52 may be moved relative to the handle 38 in order to cause the distal region 34 of the elongate shaft 32 to deflect. As an example, the deflection actuation member 52 may be moved in a direction indicated by an arrow 54 in order to cause the distal region 34 of the elongate shaft 32 to deflect. The deflection actuation member 52 may be operably coupled to the distal region 34 of the elongate shaft 32 by a pull wire. In some cases, the second electrode 50 may be operably coupled to the deflection actuation member 52 such that it is the second electrode 50 that causes the distal region 34 of the elongate shaft 32 to deflect from the biased configuration. As an example, the second electrode 50 may be a conductive member that extends essentially the length of the medical device 30. Much of the conductive member may be insulated, while a portion of the conductive member forming the visible portion of the second electrode 50 may be uninsulated in order to expose the conductive core of the conductive member.

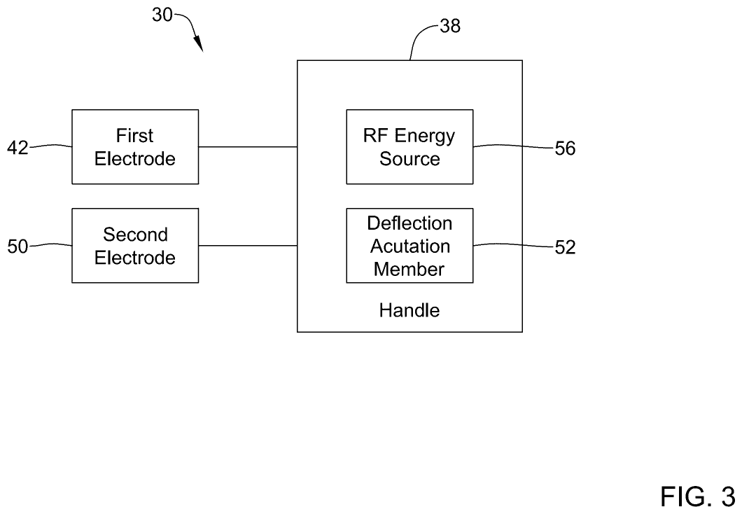

FIG. 3 is a schematic view of the illustrative medical device 30. The handle 38 is shown as including an RF energy source 56. In some cases, the RF energy source 56 may actually be disposed within the handle 38. In some cases, the RF energy source 56 may represent a set of electrical connections on the handle 38 that may allow an RF energy source that is exterior to the handle 38 to be electrically connected. In some cases, other energy sources may additionally or alternatively be used.

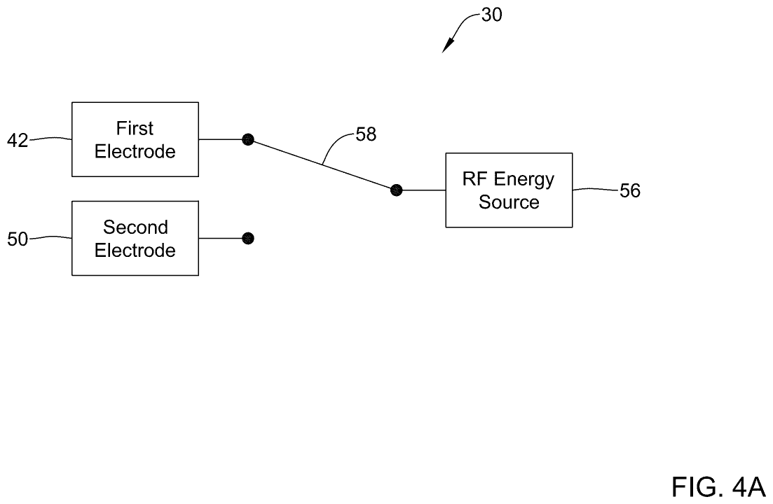

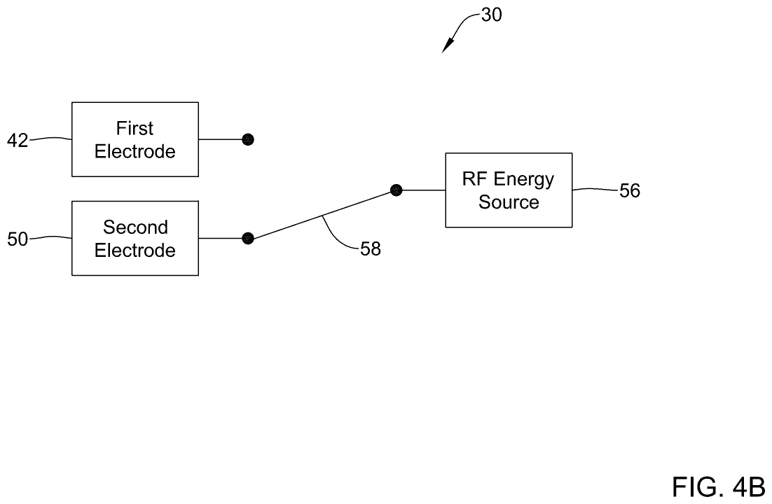

As noted, only one of the first electrode 42 and the second electrode 50 may be energized at one time. FIG. 4A shows the first electrode 42 electrically coupled to the RF energy source 56 via a switching member 58. At this time, the second electrode 50 is not electrically coupled to the RF energy source 56. FIG. 4B shows the second electrode 50 electrically coupled to the RF energy source 56 via the switching member 58. At this time, the first electrode 42 is not electrically coupled to the RF energy source 56. While the switching member 58 is shown schematically, it will be appreciated that the switching member 58 may represent an electromechanical switch. In some cases, the switching member 58 may represent an electronic switch such as part of an integrated circuit, for example.

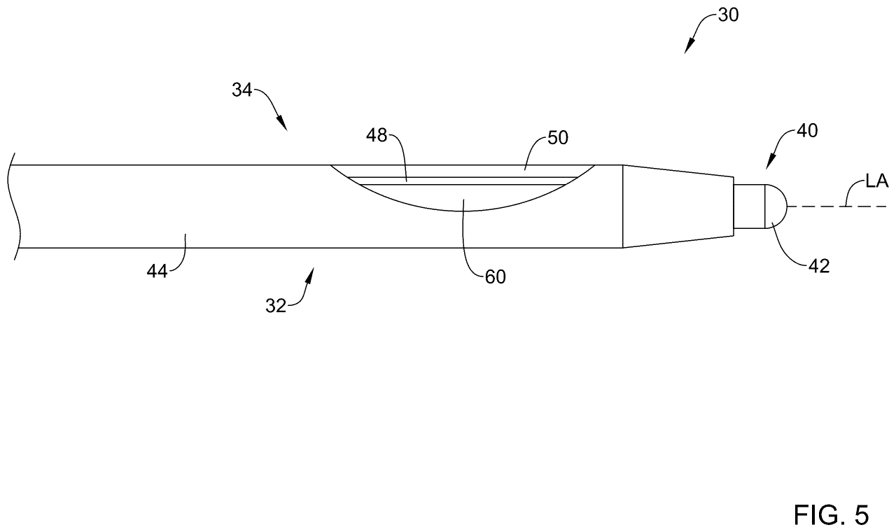

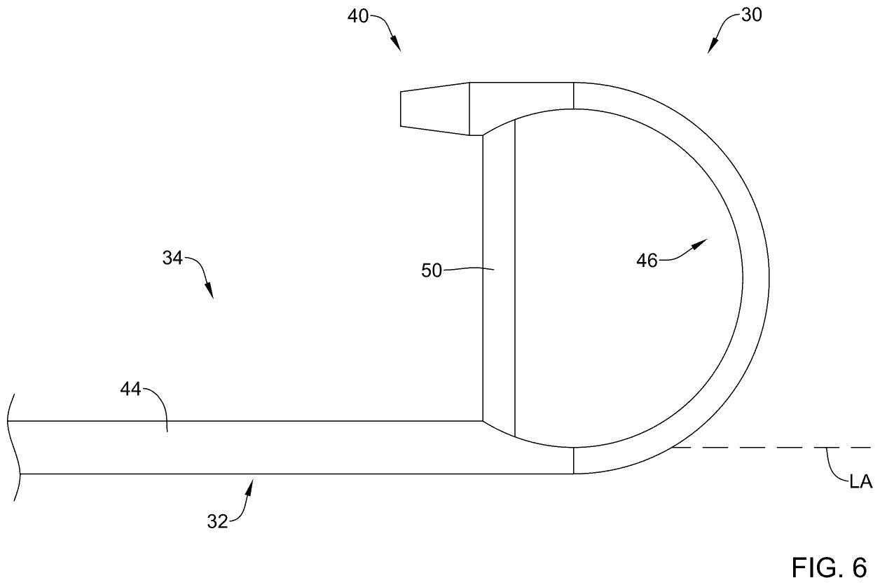

FIG. 5 is a schematic view of a portion of the medical device 30, showing the distal region 34 of the elongate shaft 32 in the biased configuration in which the distal region 34 of the elongate shaft 32 is parallel or at least substantially parallel (defined as within ten percent of parallel) with the longitudinal axis LA. FIG. 6 shows the distal region 34 of the elongate shaft 32 in a configuration in which the distal region 34 of the elongate shaft 32 is deflected from the biased configuration. In some cases, when the distal region 34 of the elongate shaft 32 is deflected away from the biased configuration, the second electrode 50 may form an angle of about 45 degrees to about 135 degrees with the longitudinal axis LA. As shown, the second electrode 50 is forming an angle of about 90 degrees with the longitudinal axis LA. When in this configuration, the medical device 30 may be pulled proximally while the second electrode 50 is energized in order to cut or lacerate tissue or other materials.

While not expressly shown, the lacerating electrode 50 may have a distal end that is secured at or near the distal end 40 of the distal region 34. Pulling on the lacerating electrode 50, such as by actuating the deflection actuation member 52, exerts a tensile force on the lacerating electrode 50 that will exert a tensile force on the distal end 40 of the distal region 34. In response, the distal region 34 of the elongate shaft 32 will deflect in response to the applied tensile force. In some cases, inclusion of the elongate slot 46 will cause the distal region 34 of the elongate shaft 32 to preferentially bend in the direction indicated in FIG. 6 because the elongate slot 46 removes material from the distal region 34 of the elongate shaft 32. The elongate shaft 32 also permits the portion of the lacerating electrode 50 passing underneath the elongate slot 46 when in the biased configuration to extend out of the elongate slot 46 when deflected from the biased configuration.

As shown in FIG. 5, an elongate member 60 may be connected to, or form part of, the first electrode 42. In some cases, the elongate member 60 may simply provide an electrical connection to the first electrode 42. In some cases, the elongate member 60 may also allow movement of the first electrode 42 relative to the distal end 40. As an example, the elongate member 60 may be used to extend the first electrode 42 to a position (as shown) in which the first electrode 42 extends distally past the distal end 40 of the elongate shaft 32. The elongate member 60 may also be used to retract or withdraw the first electrode 42 to a position in which the first electrode 42 is proximal of the distal end 40 of the elongate shaft 32, and thus may be retracted into the elongate shaft 32. It can be seen in FIG. 6 that the first electrode 42 has been retracted into the elongate shaft 32 as it no longer extends beyond the distal end 40 of the elongate shaft 32. In some cases, the elongate member 60 may be withdrawn sufficiently to position the first electrode 42 proximal of the elongate slot 46, as this can make it easier to deflect the distal region 34 of the elongate shaft 32.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some cases, the medical device 30 may include echogenic features that are capable of reflecting sound waves. This can be beneficial for imaging with ultrasound modalities such as transesophageal echocardiography and intracardiac echocardiography, for example. Echogenic features may, for example, include a braid within the elongate shaft 32, or various coiled features. In some cases, the lacerating electrode 50 may be either a cable or a solid core wire. Using a cable for the lacerating electrode 50 can provide an echogenic benefit over using a solid core wire for the lacerating electrode 50 with respect to visualization relative to anatomic structures.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device for cutting valve leaflets, the medical device comprising:
an elongate shaft extending proximally from a deflectable distal region, the deflectable distal region including a distal end and having a biased configuration;
a piercing electrode disposed relative to the distal end, the piercing electrode adapted to pierce through a valve leaflet; and
a lacerating electrode spaced and electrically insulated from the piercing electrode, the lacerating electrode adapted to lacerate the valve leaflet subsequent to the piercing electrode piercing the valve leaflet;
wherein the piercing electrode and the lacerating electrode are independently energized.

2. The medical device of claim 1, further comprising a handle secured to a proximal region of the elongate shaft.

3. The medical device of claim 2, wherein the piercing electrode is electrically coupled with the handle such that the piercing electrode can be selectively coupled with an RF energy source.

4. The medical device of claim 2, wherein the lacerating electrode extends proximally to the handle and is electrically coupled with the handle such that the lacerating electrode can be selectively coupled with an RF energy source.

5. The medical device of claim 2, wherein the handle further comprises a deflection actuation member.

6. The medical device of claim 5, wherein the lacerating electrode is coupled with the deflection actuation member such that actuating the deflection actuation member provides a tensile force on the lacerating electrode that causes the distal region to deflect from its biased configuration.

7. The medical device of claim 1, wherein the biased configuration corresponds to the lacerating electrode being at least substantially parallel with the deflectable distal region

8. The medical device of claim 1, further comprising an elongate slot formed within an outer surface of the deflectable distal region.

9. The medical device of claim 8, wherein a portion of the lacerating electrode passing the elongate slot remains within the lumen when the distal region is in the biased configuration.

10. The medical device of claim 8, wherein the portion of the lacerating electrode passing the elongate slot extends outside of the lumen when the distal region is deflected from the biased configuration.

11. A medical device, comprising:
an elongate shaft extending proximally from a distal region, the distal region including:
a distal end; and
an elongate slot extending axially along an outer surface of the distal region and terminating proximally of the distal end;
the distal region deflectable from a biased configuration;
a first electrode disposed relative to the distal end; and
a second electrode spaced from the first electrode, the second electrode remaining within the elongate slot when the distal region is in the biased configuration, the second electrode extending out of the elongate slot when the distal region is deflected from the biased configuration;
wherein only one of the first electrode and the second electrode are energized at a time.

12. The medical device of claim 11, wherein providing a tensile force on the second electrode causes the distal region to deflect from the biased configuration.

13. The medical device of claim 11, wherein the biased configuration corresponds to the second electrode being at least substantially parallel with a longitudinal axis of the elongate shaft.

14. The medical device of claim 11, wherein the deflectable region is adapted to be deflected to a configuration in which the second electrode extends at an angle between 45 degrees and 135 degrees with respect to the longitudinal axis of the elongate shaft.

15. The medical device of claim 11, wherein the second electrode is not energized when the first electrode is energized.

16. The medical device of claim 11, wherein the first electrode is not energized when the second electrode is energized.

17. The medical device of claim 11, wherein the first electrode is adapted to form a hole within a valve leaflet that the distal region can be advanced through.

18. The medical device of claim 17, wherein the second electrode is adapted to be pulled proximally through the valve leaflet subsequent to advancing the distal region through the hole in order to lacerate the valve leaflet.

19. A medical device, comprising:
an elongate shaft extending proximally from a distal region to a proximal region, the distal region including:
a distal end; and
an elongate slot extending axially along an outer surface of the distal region and terminating proximally of the distal end;
the distal region deflectable from a biased configuration;
a first electrode disposed relative to the distal end; and
a second electrode spaced from the first electrode, the second electrode remaining within the elongate slot when the distal region is in the biased configuration, the second electrode extending out of the elongate slot when the distal region is deflected from the biased configuration; and
a handle secured to the proximal region, the handle adapted to selectively connect only one of the first electrode and the second electrode to a source of RF energy at a time.

20. The medical device of claim 19, wherein the handle further comprises a deflection actuation mechanism that is adapted to apply a tensile force to the second electrode.
